(19) United States
(12) Patent Application Publication
(10) Pub. No.: US 2006/0233739 A1
(43) Pub. Date: Oct. 19, 2006

(54) TREATMENT OF MUCOCUTANEOUS DISORDERS THROUGH REVERSING CHRONIC IMFLAMMATION AND BARRIER DISRUPTION

(76) Inventors: CARL R. THORNFELDT, BOISE, ID (US); EDWARD K. PASTOR, BOISE, ID (US)

Correspondence Address: DYKAS, SHAVER & NIPPER, LLP, P.O. BOX 877, BOISE, ID, 83701-0877, US

(21) Appl. No.: 11/426,691

(22) Filed: Jun. 27, 2006

Related U.S. Application Data
(63) Continuation-in-part of application No. 10/991,227, filed on Nov. 16, 2004.
(60) Provisional application No. 60/524,962, filed on Nov. 24, 2003.

(51) Int. Cl. A61K 8/97; A61K 33/26; A61K 31/28; A61K 31/69; A61K 33/22; A61K 33/32; A61K 36/889; A61K 36/45; A61K 36/8962

(52) U.S. Cl. 424/74; 424/641; 424/646; 424/732; 424/727; 424/754; 514/492; 514/64; 424/659

(57) ABSTRACT

Topical administration for humans of formulations selected from one or more certain extracts of fruit and/or seeds of berries, date palm, onion and saw palmetto and one or more metallic moieties of metal selected from boron, vanadium and molybdenum. These formulations treat diseases and conditions including visible extrinsic skin aging, eczematous, papulosquamous, follicular, hyperpigmentation and cornification disorders and keratoses.

### PRIORITY

This application is a continuation-in-part of copending patent application Ser. No. 10/991,227, filed Nov. 16, 2004, having the same title and inventors, which itself claimed the priority date of the provisional application entitled Treatment of Mucocutaneous Disorders Through Reversing Chronic Inflammation and Barrier Disruption filed by Thornfeldt, et al. on Nov. 24, 2003 with application Ser. No. 60/524,962, the disclosures of which are incorporated by reference.

### BACKGROUND OF THE INVENTION

1. Field of the Invention

The present invention generally relates to dermatology, cosmetology, esthetics and skin care, and more particularly relates to topical compositions for the treatment of skin aging, common inflammatory diseases of the skin and mucous membranes and keratoses.

2. Background Information

Many recent discoveries in skin science suggest visible skin aging, certain common skin diseases and keratoses are primarily induced by chronic inflammation and a disrupted permeability barrier of which about 80% is caused by environmental insults. This disrupted permeability barrier of the skin itself activates cascades of biologic response modifiers which drive chronic inflammation. In addition, many of the normal skin care regimens including shaving, cleansing and applying exfoliating products further magnify the inflammation and barrier disruption. Visible skin aging (extrinsic aging) is characterized by fine lines, wrinkles, atrophy and dyspigmentation and premalignant neoplasia.

Common skin diseases characterized by chronic inflammation and abnormal permeability barrier function include: eczematous dermatitides, papulosquamous disorders of the skin, disorders of cornification, follicular inflammatory diseases, extrinsic skin aging, sebaceous hyperplasia, actinic/senile comedos, hyperpigmented lesions and keratoses.

Eczematous dermatitides are the most common group of skin diseases. Atopic dermatitis affects about thirty (30) percent of infants and children, and ten (10) percent of the U.S. population overall. It is characterized by the “itch that rashes” being distributed on flexural surfaces, head and neck. Seborrheic dermatitis may occur on the scalp, eyebrows, eyelids, ears, paranasal and intertriginous sites but usually is asymptomatic. It sometimes afflicts the middle of the chest or back. Contact dermatitis due to exposure to allergenic or irritating agents is also eczematous. Eczematous diseases are not curable unless the offending agent in contact dermatitis is completely avoided.

Papulosquamous disorders of the skin are characterized by scaly papules and plaques. The most common type is psoriasis. The skin becomes inflamed, producing red, thickened areas with silvery scales, most often on the scalp, elbows, knees and lower back. In the United States 2.1% of the population has psoriasis. It is not curable.

Disorders of cornification include among others, the group of ichthyoses. These diseases occur when normal keratinocyte cornification and desquamation does not occur. The ichthyoses include a variety of diseases with “fish scale” appearing skin surface that is resistant to moisturizers. These diseases afflict over one million people.

Follicular inflammatory diseases include acne and rosacea among others. Acne afflicts five to ten percent (5-10%) of adults and is characterized by abnormal cornification in the follicle orifice associated with sebaceous glandular hyperplasia. Rosacea is a vascular dilation disease that affects over ten percent (10%) of white adults. It is usually first noticed as a flushing or subtle redness on the cheeks, nose, chin or forehead that tends to worsen over time and produce chronic pustules and papules with enlargement of blood vessels, sebaceous glands and the nose (rhinophyma). These diseases are only suppressed by therapy, although many acne patients treated with isotretinoin develop prolonged remissions.

Actinic keratoses are common pre-malignant tumors usually on sun exposed body sites due to years of environmental damage primarily from UV radiation. Progression to squamous cell cancer ranges 2.7-16%. Benign keratoses includes seborrheic and stucco keratoses.

Sebaceous hyperplasia is a hypertrophy of the entire sebaceous gland developing into distinct visible benign neoplasms. The lesions are characterized by yellowish centrally umbilicated papules and less frequently nodules on the forehead, nose and cheeks of usually middle-aged and elderly people.

A clinical characteristic of extrinsic aging that is often a challenge to treat is actinic/senile comedones (ASC). These comedones may progress to follicular cysts and yellowish nodules usually distributed on the lateral and inferior aspects of the periorbital area. The constellation of nodules, cysts and comedones is known as Favre Racouchout (FR) disease. It is reported to afflict 6% of people 50 years of age and older, predominantly Caucasians with outdoor lifestyles and/or occupations.

Extrinsic skin aging results from environmental factors that damage epithelial surfaces, producing characteristic changes. Ultraviolet radiation accounts for about 85% of the damage, but smoking and pollutants are potent contributors. The visible changes of extrinsic aging consist of fine lines, wrinkles, roughness, atrophy, laxity, fragility, furrows, comedones, sallow, dull color, telangectasias, poor wound healing, lentigines, mottled hyperpigmentation and benign premalignant and malignant neoplasia. Skin cancer incidence has reached epidemic proportions despite potent sunscreens with sun protection factor exceeding 15 have been marketed for over 15 years.

There is also reduced stratum corneum permeability barrier as measured by transepidermal waterloss (TEWL), reduced immune surveillance and chronic inflammation. [See Bolognia J, Jorizzo J, Rapini R eds. Dermatology. Mosby/Elsevier, London 2003: 125, 175, 199, 215, 219, 777-780, 2357, 2358.] Emollients only impact the fine lines and roughness in extrinsic aging by “filling in crevasses” and improving dehydration fine lines. The benefit is not long lasting-only up to 2 days. The “best method” for long lasting true wrinkle reduction is restoration of lost collagen and ground substance and normalizing elastin fibers in the dermis. [See: DelRosso J., Cosmeceutical moisturizers in Cosmeceuticals. Draelos Z. ed. Elsevier/Saunders, Philadelphia, Pa. 2005: 97, 100, 101 and Draelos Z. Wrinkles and fine lines in Cosmeceuticals. Draelos Z ed. Elsevier/Saunders, Philadelphia, Pa. 2005: 159, 160.] Emolliency does not correlate with improved permeability barrier function as measured by reduced TEWL. Other cutaneous conditions having dehydration fine lines and roughness include contact damage from harsh cleaners, astringents, certain medications, dysfunction due to underlying hypothyroidism, diabetes or metabolic diseases as well as lipid lowering prescription medicines. Rough skin also occurs in genetic conditions including any of the twenty-one ichthyotic diseases, disorders of cornification, psoriasis and other papulosquamous and eczematous diseases including atopic and seborrheic dermatitis as well as multiple types of pityriasis. These together afflict ¼ of Americans. [See: Duke, James A. Handbook of Energy Crops: Phoenix dactylifera L. 1983. Retrieved from the Internet: <http://www.hort.purdue.edu/newcrop/duke_energy/Phoenix_{—}dactylifera.html>.] Emollients themselves do not produce resolution of any signs of visible extrinsic skin aging except dehydration fine lines.

A variety of superficial hyperpigmentation abnormalities occur with extrinsic skin aging including mottled hyperpigmentation, poikiloderma of civatte, lentigines and ephelids. It also occurs secondary to other cutaneous conditions especially inflammatory processes such as active keratoses. The current nonprescription therapies are not ideal. The prescription therapies containing several active ingredients are effective in removing the pigment but tend to induce significant cutaneous atrophy and activate contact irritation reactions.

Treatments for these conditions are sometimes not effective, not tolerated by certain individuals, or associated with one or more significant side effects that limit their use. Additional new treatments for many of these cutaneous and mucosal diseases characterized by localized chronic inflammation and compromised barrier permeability function of the skin are needed.

Current prescription, over the counter and cosmeceutical treatments for skin aging frequently induce acute and chronic inflammation, increase sensitivity to ultraviolet light among other adverse reactions that limit effectiveness. Moreover, there are numerous nonprescription skin care products claiming to be effective, yet very few have performed controlled clinical studies demonstrating efficacy and safety. It is thus readily apparent that there is a great need in the art for new and effective treatment products for age and environmental insult related skin changes, disorders and diseases.

The present invention addresses these needs in a novel manner. Additional advantages and novel features of the invention will be set forth in part in the description which follows and in part will become apparent to those skilled in the art upon examination of the following or may be learned by practice of the invention. The advantages of the invention may be realized and attained by means of the instrumentalities and combinations particularly pointed out in the appended claims.

### SUMMARY OF THE INVENTION

The present invention is based on the surprising discovery that the administration of a topical composition comprising complexes that reverse and chronic inflammation while optimizing permeability of a compromised mucocutaneous barrier is unexpectedly effective in the treatment of extrinsic skin aging signs and common inflammatory diseases of the skin, mucous membranes and keratoses.

As such, one embodiment of the present invention provides a method of treating skin and/or mucous membrane disorders characterized by chronic inflammation and a disrupted permeability barrier in a human or domesticated mammal via the administration of a topical composition to the skin and mucosa surface. The topical composition is formulated using one or more selected from each of two groups: (1) certain extracts of the berry or fruit and/or seeds of date palm (Phoenix dactylifera) fruit, bilberry (Vaccinium myrtillus), elderberry (Sambucus nigra), cranberry (Vaccinium macrocarpon), blueberry (Vaccinium angustifolum), saw palmetto (Serenoa repens), and/or onion (Allium cepa) bulb, and (2) certain metallic moieties of metal comprising boron, molybdenum and vanadium and their chelates, esters and salts thereof. Use of the phrases “date palm” and “date palm fruit” herein is intended to include date palm fruit and/or seeds.

The preferred boron donors are sodium borate, boric acid, boron glycinate, boron gluconate, zinc borate and calcium methaborate. The preferred molybdenum donors include chelates such as citrate, malate and aspartate and ammonium molybdate. The preferred vanadium donors include vanadyl sulfate, vanadium aspartate, sodium vanadate and orthovanadate.

It is preferred that the extracts are procured via one of the following methods: (a) pressing the fruit and seeds until all the liquid has been extruded resulting in a juice; (b) 100 grams of macerated fruit deposited in 1 liter of water and boiling until 50% of the volume remains (about 1 hour) then filter (this is the preferred method for the berries); and (c) 100 grams of macerated fruit and crushed seeds deposited in 70% ethanol in a 1:3 ratio by volume for 10 days then filter (this is the preferred process for date palm, saw palmetto and onion).

In one embodiment, the mucocutaneous abnormal condition is visible skin aging. The mucocutaneous diseases include, but are not limited to: actinic (solar), stucco and seborrheic keratoses; an eczematous dermatitis selected from the group of, among others, seborrheic and atopic dermatitis and contact dermatitis; a papulosquamous disease such as psoriasis; a disorder of cornification selected from the group of ichthyoses; a follicular disease selected from the group of, among others, acne, rosacea, sebaceous hyperplasia and actinic/senile comedos; and/or a hyperpigmentation disorder selected from the group of mottled hyperpigmentation, lentigines, ephelids, melasma, nevus spilus and superficial post inflammatory hyperpigmentation.

The composition is in a suitable formulation for topical administration such as a gel, an aerosol, an ointment, a cream, an emollient, an emulsion, a juice, a syrup, a tincture, a tea, a galenic extract, a lotion, a powder, a solution, a suspension, a spray, a paste, an encapsulation, a microsponge, an oil and/or a foam.

These and other aspects will become more apparent when read with the accompanying detailed description, which follows.

Further, the purpose of the foregoing Abstract is to enable the United States Patent and Trademark Office and the public generally, and especially the scientists, engineers, and practitioners in the art who are not familiar with patent or legal terms or phraseology, to determine quickly from a cursory inspection the nature and essence of the technical disclosure of the application. The Abstract is neither intended to define the invention of the application, which is measure by the claims, nor is it intended to be limiting as to the scope of the invention in any way.

Still other advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

The present invention provides a method of treating skin and/or mucous membrane disorders characterized by chronic inflammation and a disrupted permeability barrier in a human or domesticated mammal via the administration of the topical composition to the skin and mucosa surface and the formulation of the topical composition(s) itself. The preferred topical composition is formulated using one or more selected from each of two groups: (1) certain extracts of the berry or fruit and/or seeds of date palm (Phoenix dactylifera), bilberry (Vaccinium myrtillus), elderberry (Sambucus nigra), cranberry (Vaccinium macrocarpon), blueberry (Vaccinium angustifolum), saw palmetto (Serenoa repens) and/or onion (Allium cepa) bulb, and (2) certain metallic moieties of metal comprising boron, molybdenum and vanadium and their chelates, esters and salts thereof.

The preferred boron donors are sodium borate, boric acid, boron glycinate, boron gluconate, zinc borate and calcium methaborate. The preferred molybdenum donors include chelates such as citrate, malate and aspartate and ammonium molybdate. The preferred vanadium donors include vanadyl sulfate, vanadium aspartate, sodium vanadate and orthovanadate.

These extracts procured via one of the following methods: (a) pressing the fruit and seeds until all the liquid has been extruded resulting in a juice; (b) 100 grams of macerated fruit deposited in 1 liter of water and boiling until 50% of the volume remains (about 1 hour) then filter (the preferred berry extraction method); and (c) 100 grams of macerated fruit and crushed seeds deposited in 70% ethanol in a 1:3 ratio by volume for 10 days then filter (the preferred date palm, saw palmetto and/or onion extraction method).

Date palm (Phoenix dactylifera) provides a medicinal fruit that has been used in Indian medicine to treat inflamed wounds, but has no historical folk medicine use for treatment of any mucocutaneous disease or condition. The active compounds in the ethanolic extract of the fruit consist of saccharose, oligomeric proanthocyanidins, polyphenols and piperidines. Phytoestrogens are present in the ethanolic extract from the seeds. There are no reported adverse reactions with any use.

Duke, in Handbook of Energy Crops, states date extract functions as an emollient. Duke also states folk medicine uses date palm (Phoenix dactylifera) to treat cancer and induce longevity, but no statements of use in treatment of inflammatory mucocutaneous disorders are made. Moreover, no clinical trials proving scientific validity are documented. [See: Duke, James A. Handbook of Energy Crops: Phoenix dactylifera L. 1983. Retrieved from the Internet: <http://www.hort.purdue.edu/newcrop/duke_energy/Phoenix_{—}dactylifera.html>]. One reference states Indian medicine used date palm honey for inflamed wounds. [See: LaGow, B., Chief ed., PDR for Herbal Medicines, 3rd Ed., Thomson PDR, Montvale, N.J. 2004: 254.] Another major source states there is insufficient scientific evidence for date palm to publish it. [See: Jellin, J., ed-in-chief, Natural Medicines Comprehensive Database, 8th ed., Therapeutic Research Faculty, Stockton, Calif., 2006: 418.]

Saw palmetto (Serenoa repens) is a proven therapy for benign prostatic hypertrophy. Folk medicine uses included anti-inflammatory, antiseptic, cancer, asthma and stimulate scalp hair growth, anti-androgen, anti-proliferative and anti-estrogenic effects. There were no documented contact reactions. [See: Jellin, J., ed-in-chief, Natural Medicines Comprehensive Database, 8th ed., Therapeutic Research Faculty, Stockton, Calif., 2006: 1115-1117.]

Elderberry (Sambucus nigra) has medicinal fruit which yields anthocyanadins, tannins and essential oil. These provide antiviral, anti-inflammatory and immuomodulatory effects. Fold medicine uses elderberry extracts to treat allergic rhinitis, sinusitis, cancer and constipation. It “possibly” is an effective treatment for influenza. No contact reactions were reported. [See: Jellin, J., ed-in-chief, Natural Medicines Comprehensive Database, 8th ed., Therapeutic Research Faculty, Stockton, Calif., 2006: 464-465.]

Onion (Allium cepa) contains a medicinal bulb from which are extracted alkyl cysteine sulfoxides (alliins), fructosans, saccharose, flavonoids diphenylamine, sulfur compounds, ascorbic acid and steroid saponins. These provide broad-spectrum antibacterial and antiallergic effects. Onion is used in Indian medicine for wounds and Chinese medicine for parasite, fungal and bacterial infections. It is approved by the Commission E of Germany for oral inflammation. Onion occasionally produces allergic contact dermatitis. [See: Jellin, J., ed-in-chief, Natural Medicines Comprehensive Database, 8th ed., Therapeutic Research Faculty, Stockton, Calif., 2006: 938-939.]

Bilberry (Vaccinium myrtillus) has medicinal berries and leaves. An aqueous extract acquired by boiling berries in water provides malic and citric fruit acids, catechins, anthocyanoides, polyphenols, flavonoids such as quercitrin, iridoids, chlorogenic acid and pectins. These actives provide anti-inflammatory, anti-edema, astringent, anti-oxidant, antiseptic, wound healing, antineoplastic and antihistaminic effects. It also stimulates glycosaminoglycan synthesis. Numerous randomized prospective controlled clinical trials proved its therapeutic activity for several eye disorders as well as dysmennorhea. Oral bilberry effectively treated symptomatic varicose veins and symptomatic venous insufficiency. No scientific trials using a topical composition have been published. The Commission E has approved bilberry for treatment of oral inflammation. Unproven folk medicine uses this herb for wound healing, skin ulcers and hemorrhoids. No reactions have been reported. [See: Jellin, J., ed-in-chief, Natural Medicines Comprehensive Database, 8th ed., Therapeutic Research Faculty, Stockton, Calif., 2006: 133-134.]

Blueberry (Vaccinium angustifolium) has medicinal fruit that contains ascorbic acid, oligomeric proanthocyanidins, and anthocyanins, which provide anti-oxidant, antineoplastic and bacteriostatic effects. Blueberry's high concentration of manganese allows its use as a negative contrast agent for magnetic resonance imaging of the gastrointestinal tract. It has no reported adverse effects. [See: Jellin, J., ed-in-chief, Natural Medicines Comprehensive Database, 8th ed., Therapeutic Research Faculty, Stockton, Calif., 2006: 186-187.]

Cranberry (Vaccinium macrocarpon) is used to treat scurvy, fever, cancer and as an anti-oxidant and antiseptic against a broad range of bacteria as well as periodontal disease. The major active ingredients include oligomeric proanthocyanidins and salicylates. There are no scientific studies documenting efficacy in any mucocutaneous disease. There are no reported mucocutaneous adverse reactions. [See: Jellin, J., ed-in-chief, Natural Medicines Comprehensive Database, 8th ed., Therapeutic Research Faculty, Stockton, Calif., 2006: 402-403.].

Boron as boric acid and borox is used therapeutically as a mild topical antiseptic but no other cutaneous conditions. Absorption through intact skin from a 10% aqueous solution is too minimal to cause systemic toxicity. When applied to damaged (burned or abraded) skin involving ≧4% of body surface area, much greater penetration can occur resulting in CNS depression and death. Boron hydrides (boranes) are highly toxic. [See: Guy, R., et al., Metals and the Skin. Marcel Dekker, New York, N.Y., 1999: 87-90.] Boron is important for membrane function, mineral metabolism and increases estradiol, which reverses cutaneous atrophy. [See: Jellin, J., ed-in-chief, Natural Medicines Comprehensive Database, 8th ed., Therapeutic Research Faculty, Stockton, Calif., 2006: 194-196].

Molybdenum is an essential trace element vital to animal biochemistry. High soil levels are associated with increased gout. This mineral frequently causes systemic and contract allergic reactions, activating both cellular and humoral immunity. It inhibits crosslinking of collagen. Molybdenum is closely associated with copper and iron metabolism and enzymatic redox processes for aldehydes, sulfites, nitrates and molecular nitrogen. Molybdenum chloride is highly toxic. [See: Guy, R., et al., Metals and the Skin. Marcel Dekker, New York, N.Y., 1999: 267-270.]

Vanadium is an essential nutrient in humans functioning in thyroid hormone metabolism, bone growth and mimics epidermal and fibroblast growth factors and insulin. This mineral inhibits adenosine triphosphatases, phosphatases, sulfur containing amino acid metabolism such as cystine, cholesterol synthesis and phosphoryl transfer enzymes. Vanadium toxicity includes a green discolored tongue. Vanadium pentaxide is toxic systemically and topically. Vanadium is both cellular and humoral immunity toxic. [See: Jellin, J., ed-in-chief, Natural Medicines Comprehensive Database, 8th ed., Therapeutic Research Faculty, Stockton, Calif., 2006: 1258-1260; and Guy, R., et al. Metals and the Skin. Marcel Dekker, New York, N.Y., 1999: 381-384.]

The term “patient” is used herein to refer to an organism in need of treatment of one or more condition, abnormality and/or disease of the skin and/or mucous membrane. Preferably, a patient is a human or a domesticated animal. Note: use of the terms “condition,” “abnormality,” and/or “disease” are intended to be generally interchangeable within this disclosure, in that reference to a “condition” is likewise intended to include “abnormalities” and/or “diseases,” and vice versa.

The terms “treatment,” “therapy” and the like include, but are not limited to, changes in the patient's status. The changes can be either subjective or objective and can relate to features such as symptoms or signs of the disease/abnormality/condition being treated. For example, if the patient notes decreased itching, reduced discomfort, size or visibility of skin lesions, then successful treatment has occurred. Preventing the deterioration of a patient's status is also included by the term. Therapeutic benefit included any of a number of subjective or objective factors indicating a response of the condition being treated as discussed herein.

“Therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation, which causes a substantial improvement in a disease/condition/abnormality when applied to the affected area(s) repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

The term “cosmeceutical composition” means a composition suitable for cosmeceutical use in or upon a human or domesticated mammal subject. Such a composition generally comprises an effective amount of an active agent that is categorized as an excipient that improves appearance and modulates certain superficial epithelial membrane physiological functions in non-prescription formulations.

The berry fruits include extracts of fruit and/or seeds of, but are not limited to date palm, elderberry, bilberry, blueberry and cranberry. Additional herbs of this invention included saw palmetto and onion. The concentration of one or more of these each ranges from 0.001% to 51.00% by weight/weight with preferred concentration from 0.05% to 20.00%.

The second active is selected from metal comprising boron, molybdenum and vanadium and their chelates, esters and salts thereof. The boron donors are sodium borate, boric acid, boron glycinate, boron gluconate, zinc borate and calcium methaborate. The molybdenum donors include chelates such as citrate, malate and aspartate and ammonium molybdate. Vanadium donors include vanadyl sulfate, vanadium aspartate, sodium vanadate and orthovanadate. The concentration preferably ranges from 0.01% to 51.00% by weight/weight with a preferred concentration of 0.25% to 20.00%.

The present formulations can be applied to treat an existing disease or condition, or can be used “prophylactically.” In prophylactic applications, compositions containing the present compounds can be administered to a patient that does not already suffer from disease lesions in order to enhance the patient's resistance or to retard the progression of disease or condition. Such an amount is defined as a “prophylactically effective dose or amount.”

In a preferred embodiment, mucocutaneous conditions and diseases characterized by chronic inflammation and a disrupted permeability barrier such as skin aging, eczematous and papulosquamous diseases, acne, rosacea, actinic/senile comedos, mottled hyperpigmentation, lentigines, ichthyoses and keratoses, are effectively treated with topical administration of formulations comprising the ethanolic extract of date palm fruit and sodium borate.

In the most preferred embodiment, visible skin aging, seborrheic, atopic and contact dermatitis, psoriasis, ichthyoses, acne, rosacea, sebaceous hyperplasia, actinic/senile comedos, mottled hyperpigmentation, lentigines and keratoses are effectively treated topical administration of a formulation comprising the ethanolic extract of date palm fruit, onion, sodium borate and zinc borate.

The present compositions can be used to treat any symptom associated with any of these diseases or conditions, such as swelling, redness, itching, pimples, pustules, crusts, scabs, dryness, burning, oozing, fluid, pus, discharge, rashes, small cysts, milia, disfiguration, scaling, dandruff, inflammatory and non-inflammatory papules, plaques, lesions, thickenings, shedding, bumps, flaking, bleeding, tenderness, cuts, scratches, irritation, swelling, blebs, vesicles, bullae, elevations, scarring, fine lines, wrinkling, mottled hyperpigmentation, lentigines, freckling, yellowing (sallow), violaceous, blood vessel dilation, open and closed comedomes, photosensitivity, dyspigmentation, atrophy and others.

A number of suitable formulations for use in the present invention are found in Remington's Pharmaceutical and Cosmeceutical Sciences (Mack Publishing Company, Philadelphia, Pa., 17^{th} ed., 1985), which is incorporated herein by reference. Moreover, for a brief review of methods for drug delivery, see Langer, Science 249: 1527-1533 (1990), which is also incorporated herein by reference. The pharmaceutical compositions described herein can be manufactured in a manner that is known to those of skill in the art, i.e., by means of conventional mixing, dissolving, levigating, emulsifying, encapsulating, entrapping or lyophilizing processes. It will be appreciated that the present methods and excipients are merely exemplary and are in no way limiting.

These pharmaceutical and cosmeceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, pluronic polyols, glycerol monolaurate, sulfoxides, terpenes (e.g., menthol), amines, amides, alkanes, alkanols, ORGELASE, water, calcium carbonate, calcium phosphate, various sugars, starches, cellulose derivatives, gelatin, and polymers such as polyethylene glycols.

Dosage amount and interval may be adjusted individually to provide levels of the active compound, which are sufficient to maintain therapeutic effect. One having skill in the art will be able to optimize therapeutically effective local dosages without undue experimentation.

The preferred anti-inflammatory agents for inclusion into the cosmeceutical products of this invention include, but are not limited to, nonprescription topical corticosteroids and pyrithiones, menthol, phenol and resorcinol.

This invention is a surprising discovery for several reasons. First, the strong toxicity potential of the metals including contact sensitization of molybdenum and vanadium. Boron is systemically absorbed from open wounds occasionally inducing central nervous system (CNS) depression and rarely death. [See: Guy, R., et al. Metals and the Skin. Marcel Dekker, New York, N.Y., 1999: 87-90, 267-270, 381-384.] Second, none of the botanical extracts and minerals are listed in either recent major text of natural medicines to reverse skin aging with oral or topical administration. Third, the common contact allergic and irritant hand dermatitis to onion. Fourth, the major functional components of the fruit/berry extracts are dominated by aqueous factors. None have ever been documented in prospective controlled clinical trials with topically applied compositions to produce a measurable therapeutic effect in a lipophilic stratum corneum environment. Controlled trials with bilberry and saw palmetto berry extracts have proven therapeutic efficacy with orally administered compositions for vascular and prostatic diseases, not cutaneous disorders. [See: LaGow, B., Chief ed., PDR for Herbal Medicines, 3rd Ed., Thomson PDR, Montvale, N.J. 2004: 76-80, 254, 606-608, 707-710 and 1-128, and Jellin, J., ed-in-chief, Natural Medicines Comprehensive Database, 8th ed., Therapeutic Research Faculty, Stockton, Calif., 2006: 133, 134, 186, 187, 194-196].

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiment. As will be apparent to persons skilled in the art, modification and adaptations to the above-described invention can be made without departing from the spirit and scope of the invention, which is defined and circumscribed by the appended claims.

### EXAMPLE I

A first formulation for topical administration to humans comprises:

| | |
|---|---|
| Ethanolic extract of date palm fruit and seeds: | 1.0-5.0% |
| Sodium Borate: | 0.01-0.10% |
| Eucerin ® Lotion by Biersdorf: | qsad 100 grams |

### EXAMPLE II

A second formulation for topical administration to humans comprises:

| | |
|---|---|
| Ethanolic extract of date palm fruit: | 1.0-5.0% |
| Ethanolic extract of saw palmetto fruit: | 1.0-5.0% |
| Vanadyl sulfate: | 0.01-0.10% |
| Eucerin ® Lotion by Biersdorf: | qsad 100 grams |

### Study I

A controlled usage clinical study with 12 patients was conducted for 12 weeks of twice daily application of the formulation in EXAMPLE I. Dermatologists clinical assessment revealed: 34.8%—resolution of fine lines, 23.5%—resolution of wrinkles, 61.5%—resolution of roughness, and 33.5%—resolution of mottled hyperpigmentation.

### Study II

The formulation of EXAMPLE I was used twice daily for 16 weeks to 34 people afflicted with visible cheek nonhypertrophic actinic keratoses. All visible lesions (100%) resolved by completion of the study.

### Study III

The formulation of EXAMPLE I was used twice daily for 16 weeks to treat 4 patients suffering from rosacea. All inflammatory papules and pustule cleared by 9 weeks. Telangectasias decreased in size by 80%.

### Study IV

The formulation of EXAMPLE I was applied twice daily to seventeen elderly panelists to reverse sebaceous hyperplasia. After sixteen weeks, they experienced 49% reduction in the number of visible lesions, documented by board certified dermatologist investigators.

### Study V

The formulation of EXAMPLE I was applied twice daily to twenty elderly panelists to reverse actinic/senile comedones. After sixteen weeks, 56% of the visible lesions had resolved.

### Study VI

The formulation of EXAMPLE I was applied twice daily to seven elderly panelists to reverse visible premalignant keratoses. After sixteen weeks, 62% had completely cleared.

### Study VII

The formulation of EXAMPLE I was applied twice daily to twenty-three elderly panelists to reverse visible extrinsic aging parameters in the periocular area. After sixteen weeks, a 48% reduction in visible periocular wrinkles was documented.

### Study VIII

The topical formulation of EXAMPLE II was applied twice daily in a randomized manner to half the face of six middle-aged women afflicted with mild acne vulgaris and extrinsic aging, but suffer with sensitive skin. The other half of the face was treated with Eucerin® lotion (by Biersdorf) only. The composition of this invention-treated side more clear and healthier than the other side according to 5/6 of the panelists. The trained skin professional evaluators also had a superior grade on global assessment of the test product treated half face on 4/6 of the panelists.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

We claim:

1. A cosmeceutical composition for treating one or more mucocutaneous conditions in a patient having skin by topically administering to the patient a therapeutically effective amount of a cosmeceutical composition, said cosmeceutical composition comprising:
(a) at least one fruit extract selected from the group consisting of date palm fruit, bilberry, elderberry, cranberry, blueberry, saw palmetto berries and onion bulb, said fruit extract in an amount of about 0.01 to 51.00 percent by weight;
(b) at least one metallic moiety selected from the group consisting of sodium borate, boric acid, boron glycinate, boron gluconate, zinc borate, calcium methaborate, molybdenum citrate, molybdenum malate, molybdenum aspartate, ammonium molybdate, vanadyl sulfate, vanadium aspartate, sodium vanadate and orthovanadate in an amount of about 0.001 to 51.00 percent by weight; and
(c) a pharmaceutically acceptable carrier.

2. The cosmeceutical composition of claim 1, wherein said fruit extract comprises ethanolic extract of date palm fruit and said metallic moieties comprise sodium borate.

3. The cosmeceutical composition of claim 1, wherein mucocutaneous condition is selected from the group consisting of visible skin aging, atopic dermatitis, seborrheic dermatitis, contact dermatitis, psoriasis, acne, rosacea, sebaceous hyperplasia, actinic/senile comedos, mottled hyperpigmentation, lentigines and keratoses.

4. The cosmeceutical composition of claim 1, further comprising an anti-inflammatory agent, wherein said anti-inflammatory agent consists of at least one agent selected from the group consisting of pyrithiones, menthol, phenol and resorcinol.

5. A cosmeceutical composition for treating one or more mucocutaneous conditions in a patient having skin by topically administering to the patient a therapeutically effective amount of said cosmeceutical composition, said cosmeceutical composition comprising:
(a) at least one fruit extract selected from the group consisting of date palm fruit, bilberry, elderberry, cranberry, blueberry, saw palmetto berries and onion bulb, said fruit extract in an amount of about 0.01 to 51.00 percent by weight;
(b) at least one metallic moiety selected from the group consisting of sodium borate, boric acid, boron glycinate, boron gluconate, zinc borate, calcium methaborate, molybdenum citrate, molybdenum malate, molybdenum aspartate, ammonium molybdate, vanadyl sulfate, vanadium aspartate, sodium vanadate and orthovanadate in an amount of about 0.001 to 51.00 percent by weight;
(c) an anti-inflammatory agent, wherein said anti-inflammatory agent comprises at least one agent selected from the group consisting of pyrithiones, menthol, phenol, and resorcinol; and
(d) a pharmaceutically acceptable carrier.

6. The cosmeceutical composition of claim 5, wherein mucocutaneous condition is selected from the group consisting of visible skin aging, atopic dermatitis, seborrheic dermatitis, contact dermatitis, psoriasis, acne, rosacea, sebaceous hyperplasia, actinic/senile comedos, mottled hyperpigmentation, lentigines and keratoses.
